(19) United States
(12) Patent Application Publication
(10) Pub. No.: US 2003/0161825 A1
(43) Pub. Date: Aug. 28, 2003

(54) Method for reducing corrosion from copper wood preservatives

(76) Inventor: Michael Howard West, Senatobia, MS (US)

Correspondence Address: MICHAEL H. WEST, 54 SOUTH CROCKETT ROAD, SENATOBIA, MS, 38668, US

(21) Appl. No.: 10/080,253

(22) Filed: Feb. 22, 2002

(51) Int. Cl. A61K 47/00; A01N 25/00

(52) U.S. Cl. 424/126

(57) ABSTRACT

Disclosed is a method for reducing the mild steel corrosion from aqueous wood preservative solutions of copper oxide, copper carbonate, or copper hydroxide with formic, acetic, or propionic acid which comprises combining in these wood preservative solutions zinc oxide or zinc carbonate dissolved with formic, acetic, or propionic acid.

### CROSS REFERENCE TO RELATED APPLICATIONS

NOT APPLICABLE

### STATEMENT REGARDING FEDERALLY SPONSORED RESEARCH OR DEVELOPMENT

NOT APPLICABLE

### REFERENCE TO A MICROFICHE APPENDIX

NOT APPLICABLE

### BACKGROUND OF THE INVENTION

The field of endeavor to which this invention pertains relates to the treatment of wood with aqueous solutions of copper oxide, copper carbonate, or copper hydroxide for long term protection from attack by insects, decay, and mildew.

It has been known commercially in the prior art to dissolve these copper compounds in water using ammonia or short chain amines. These alkaline copper solutions are usually sufficiently free of corrosion to be used in existing mild steel wood treating equipment, and the treated wood is sufficiently free of corrosive properties to be held in place using common mild steel fasteners. However, there are many other problems associated with copper solutions in aqueous ammonia or short carbon chain amines including human health, environmental, and efficacy concerns.

It has been further known in the prior commercial art to dissolve copper oxide, copper carbonate, or copper hydroxide using inorganic acids, and to combine these in aqueous solution with chromates for fixation in wood and for corrosion inhibition. In recent years chromates have faced restrictions because of concerns for their effects on human health and the environment, and they are no longer a viable option for use in wood treating.

It has been known in the prior experimental art to dissolve copper oxide, copper carbonate, or copper hydroxide in water using formic, acetic, or propionic acids; and to employ these solutions for long term protection of wood from attack by insects, decay and mildew. Acetic acid and propionic acid are especially benign where human health and the environment are concerned; they are found in both animal and human foodstuffs. In the past these solutions have not been used commercially because they were highly corrosive to mild steel, and they were incompatible with the known chromate corrosion inhibitors.

I have now discovered that zinc oxide or zinc carbonate dissolved with formic, acetic or propionic acid is an effective corrosion inhibitor for these copper solutions without raising problems for human health or the environment. The zinc compounds do not delete from the efficacy of the copper wood preservatives, and they are economical.

### BRIEF SUMMARY OF THE INVENTION

My invention relates to a method for reducing the mild steel corrosion from aqueous wood preservative solutions of copper oxide, copper carbonate, or copper hydroxide with formic, acetic, or propionic acid which comprises combining in these treating solutions zinc oxide or zinc carbonate dissolved with formic, acetic, or propionic acid wherein the weight ratio of zinc compound to copper compound ranges from about 0.1 to about 10. It further relates to a method for producing treated wood with reduced mild steel corrosion properties which comprises treating said wood with an effective amount of preservative according to the above method. The object of my invention is to produce a commercial acidic copper wood preservative which is acceptable from the standpoint of corrosion, efficacy, economics, human health, and the environment.

### DETAILED DESCRIPTION OF THE INVENTION

The best mode for carrying out my invention is to dissolve the copper compound and the zinc compound in water with the organic acid to prepare the wood preservative solution, and to apply this preservative to wood by pressure application. The preferred embodiment of my preservative solution method comprises copper hydroxide, zinc oxide, and propionic acid. The weight ratio of zinc oxide to copper hydroxide may vary from about 0.1 to 10, and preferably about 1 to 1. Propionic acid is preferably used in slight chemical equivalent excess of the copper hydroxide plus zinc oxide. If less propionic acid is used, some of the zinc oxide will not dissolve and may cause cosmetic problems. If a large excess of propionic acid is used, corrosion inhibition will be compromised.

Examples 1, 2, and 3 illustrate wood preservative treating solutions according to my method invention. Those skilled in art will understand there are other avenues for preparing the preservative treating solution according to my method invention including the formulation of preservative concentrates. They will further understand that additional components may be included in the preservative compositions for which I teach a method. They may not recognize addition of components could effect toxicology, environment, economics, and efficacy as well as corrosion properties.

The formulations of these illustrative preservative formulations were prepared by adding the components in the order listed, and blending for 30 minutes to achieve complete copper and zinc solution according to my method invention:

### EXAMPLE 1

water 97.0 pbw

technical zinc oxide 0.5 pbw

technical copper hydroxide 0.5 pbw

technical propionic acid 2.0 pbw

### EXAMPLE 2

water 97.0 pbw

technical zinc oxide 0.1 pbw

technical copper hydroxide 0.9 pbw

technical propionic acid 2.0 pbw

### EXAMPLE 3

water 97.0 pbw

technical zinc oxide 0.9 pbw

technical copper hydroxide 0.1 pbw

technical propionic acid 2.0 pbw

The wood treating formulation in Example 4 was prepared not according to the method of my invention in order to illustrate the mild steel corrosion properties of water soluble, acidic, organic wood preservatives taught in the prior art:

### EXAMPLE 4

water 97.0 pbw

technical copper hydroxide 1.0 pbw

technical propionic acid 2.0 pbw

All four of these preparations from the examples were stored at ambient temperature for two weeks in mild steel containers. At the end of this period each container and solution was examined for iron corrosion products. In each case the zinc oxide reduced mild steel corrosion over that found in the container without zinc oxide. There were no corrosion products in the containers which held the composition from Examples 1 and 3.

I claim:

1. A method for reducing the mild steel corrosion from aqueous wood preservative solutions of copper oxide, copper carbonate, or copper hydroxide with formic, acetic, or propionic acid which comprises combining in these wood preservative solutions zinc oxide or zinc carbonate dissolved with formic, acetic, or propionic acid wherein the weight ratio of zinc compound to copper compound ranges from about 0.1 to about 10.

2. A method according to claim 1 wherein copper hydroxide is the copper compound in the wood preservative solution.

3. A method according to claim 1 wherein the acid in the wood preservative solution is propionic acid.

4. A method according to claim 1 wherein the zinc compound in the wood preservative solution is zinc oxide.
